(19) United States
(12) Patent Application Publication
(10) Pub. No.: US 2006/0148059 A1
(43) Pub. Date: Jul. 6, 2006

(54) Salt tolerant l-myo-inositol 1-phosphate synthase and the process of obtaining the same

(76) Inventors: Arunendra Nath Lahiri Majumder, Kolkata (IN); Majee Manoj, Kolkata (IN)

Correspondence Address: THE WEBB LAW FIRM, P.C., 700 KOPPERS BUILDING, 436 SEVENTH AVENUE, PITTSBURGH, PA, 15219, US

(21) Appl. No.: 10/538,423

(22) PCT Filed: Mar. 21, 2003
(86) PCT No.: PCT/IN03/00065

§ 371 Date: Jan. 30, 2006

(30) Foreign Application Priority Data
Dec. 12, 2002 (IN) 1250/DEL/02

(51) Int. Cl. C12N 9/16; C12N 1/20

(52) U.S. Cl. 435/196; 435/252.3

(57) ABSTRACT

A salt tolerant L-myo-inositol 1-phosphate synthase for Porteresia coarctata (PINO1), the nucleotide sequences and the deduced aminoacid sequence which is shown later.

### FIELD OF INVENTION

This invention relates to a salt tolerant L-myo-inositol 1-phosphate synthase and the process of obtaining the same.

### BACKGROUND OF THE INVENTION

In agricultural biotechnology a long standing goal is to improve tolerance of crop plants to environmental stress such as salinity, drought and temperature mediated dehydration all of which constitute direct osmotic stress. Once of the mechanisms by which plants respond to such abiotic stress conditions is by synthesizing non-toxic biomolecules termed compatible solutes or osmoprotectants. These compounds fall into three categories; amino acids (eg proline), onium compounds (eg glycinebetaine, dimethylsulphoniopropionate) and polyols/sugars (eg inositol, ononitol/pinitol mannitol, trehalose). Over production of any such osmoprotectant by introgression of genes encoding critical steps in the synthesis of these compounds through metabolic engineering has become the choice of biotechnologists for raising stress tolerant crop plants. Such approaches have met limited success in both pro- and eukaryotic systems. More importantly, it is imperative that the critical step for manipulation should itself encode a stress-tolerant enzyme protein.

Although metabolic engineering involving overproduction of selected osmolytes has been a choice for imparting stress tolerance phenotype in plants and other organisms, none of the systems used any stress tolerant gene/enzyme for such work. Hence, functional expression of the target gene/enzyme in the transgenic system remained unpredictable.

### OBJECTS OF THE INVENTION

An object of this invention is to produce a salt-tolerant L-myo-inositol 1-phosphate synthase gene.

Another object of this invention is to provide a process for obtaining a salt tolerant genie for inositol production.

Yet another object of this invention is to introgress the salt tolerant L-myo-inositol 1-phosphate synthase in model crop plants for its functional expression to confer ability to grow in presence of salt without decline in photosynthetic functions.

### BRIEF DESCRIPTION OF THE INVENTION

The present invention, provides a salt-tolerant L-myo-inositol 1 phosphate synthase from Porteresia coarctata.

Also provided in accordance with the present invention is a process of obtaining a salt tolerant myo-inositol 1 phosphate synthase gene comprising:
(i) isolation of a full-length cDNA for the L-myo-inositol 1-phosphate synthase gene from the leaf of Porteresia coarctata (PINO1) by reverse transcription followed by polymerase chain reaction;
(ii) sequencing of the isolated L-myo-inositol 1-phosphate synthase gene;
(iii) Cloning of the isolated full length cDNA of PINO1 in suitable bacterial expression vectors to obtain the expression plasmid construct.
Introduction of the expression plasmid construct into the bacterial host strain, E. coli BL21 (DE 3) by transformation and induction of expression of to PINO1 gene project by IPTG.

Isolation of the expressed PINO1 gene product as inclusion bodies solubilization and isolation of the active enzyme protein in a buffer containing 8M Urea, 0.5 M NaCl, 20 mM Tris-HCl, pH 7.5, 10 mM βmercaptoethanol (ME) and 2 mM phenylmethylsulphonylfluoride (PMSF) and its complete purification to homogeneity.

### DETAILED DESCRIPTION OF THE INVENTION

Cloning and sequencing of L-myo-inositol 1-phosphate synthase gene from. Porteresia coarctata (PINO1) and its comparison with that from Oryza saliva (RINO1).

A full length cDNA for the L-myo-inositol 1-phosphate synthase gene has been obtained from Porteresia coarctata (PINO1) as well as Oryza sativa (RINO1) leaf poly-A (RNA) by reverse transcription-polymerase chain reaction (RT-PCR). Total RNA was isolated from mature leaves of Oryza and Porteresia following the method of Ostrem et al (Plant Physiol. 84, 1270-1275,1987). Poly-A RNA was isolated from the total RNA by the polyAtract mRNA isolation kit (Promega) following the manufacturer's instructions. 20-30 ng of poly-A RNA was used for first stand cDNA synthesis using Superscript II RNAse H-reverse transcriptase (Life Technologists; Gibco BRL) following the manufacturer's protocol. cDNA thus synthesized was used as template for PCR amplification of the inositol synthase gene. For cloning of the full-length cDNA of inositol synthase for Oryza (RINO1) and Porteresia (PINO1), sense (5′-3′) and anti-sense (3′-5′) oligonucleotide primers were designed based on the published RINO1 sequences (GenBank accession number AB 012107) and PCR amplification was done as follows: 94-1 min[94-1.5 min; 55, 1.5 min; 72, 2 min]×32 cycles; 72, 10 mins. The amplified product was checked for the expected size (˜1.5 kb), band eluted from the gel, purified through QIAquick PCR purification kit (Qiagen) and ligated overnight at 4° C. to the pGEM T-Easy vector (Promega) following manufacturer's instructions. The ligation mixture was used for transformation of high efficiency JM109 competent cells (Promega) and transformants were selected based on blue/white selection on ampicilin/IPTG/X-gal plates grown overnight Minipreps of the plasmids were isolated from the transformants, the DNA digested with EcoR1 and the digested DNA analyzed by agarose gel electrophoresis for the expected ˜1.5 kb insert. Having confirmed the insert size, plasmid DNA was isolated from the transformants and purified through the Qiaquick purification it (Qiagen). The clones were designated as RINO1 for the gene for inositol synthase from Oryza sativa and PINO1 for the same from Porteresia coarctata.

The nucleotide sequence for each clone was determined through automated DNA sequencing. The sequencing strategy involved several cycles of sequencing of the clones by designated primers as follows:
1. First round with primers for T₇ promoter at the 5′ end and SP₆ promoter at the 3′ end.
2. Second round with primers designed at the 5′ end and the 3′ end of the gene as used for RT-PCR amplification.
3. Third round of sequencing with primers designed at about 250 base pairs downstream the start site and 250 nucleotides upstream of the stop site.

The sequencing data from each set were compiled and compared to work out the complete sequence of the L-myo-inositol 1-phosphate synthase from Porteresia coarctata (PINO1) and Oryza sativa (RINO1, GenBank accession number AB012107). The complete sequence of PINO1 is provided hereunder:

```
PINO1 Sequence:
atgttcatcgagagcttccgcgtggagagcccgcacgtgcggtacggcgcggcggagatc
  M F I E S F R V E S P H V R Y G A A E I
gagtcggagtaccggtacgacactacggagctggtgcacgagagccacgacggcgcctcg
  E S W Y R Y D T T E L V H E S H D G A S
cgctgggtcgtccgccccaagtccgtccagtaccacttcaggaccagcaccaccgtcccc
  R H V V R P K S V Q Y H F R T S T T V P
aagctcggggtcatgctcgtggggtggggcggcaacaacggctcaacgctgacggctggg
  K L G V M L V G W G G N H G S T L T A G
gtcatcgccagcagggagggaatctcatgggcgaccaaggacaaggtgcagcaagccaac
  V I A S R E G I S W A T K D K V Q Q A N
tactatggctcactcacccaggcgtccaccatcagggtaggaagctacaacggggaggag
  Y Y G S L T Q A S T I R V G S Y N G E E
atctacgcgcctttcaagagcctcctgcccatggtgaaccctgatgaccttgtgttcggg
  I Y A P F K S L L P M V N P D D L V F G
ggctgggacattagcaacatgaacctggctgatgctatgaccagggccaaggtgctggac
  G W D I S N M N L A D A M T R A K V L D
attgatctgcagaagcagcttaggccttacatggagtcctggtgcctctccctggcatct
  I D L Q K Q L R P Y M E S W C L A L A S
atgatcccgacttcatcgccgctaaccagggatcccgcgcgaacaatgtcatcaagggaa
  M I P T S S P L T R D P A R T M S S R E
ccaagaaggagcagatggggcagatcatcaaaggacatcagggagttcaaggaaaataac
  P R R S R W G R S S K D I R E F K E N N
aaaatggacaaggcggtggtgttgtggactgcaaacactgaaaggtacaacaattgtctg
  K M D K A V V L N T A N T E R Y N N C L
tgtttgggcttaatgaccaatggaaaaccttctgcgtctgtggacaggaaccaggcggag
  C L G L M T N G K P S A S V D R S Q A E
atatcgccatcgacattgtattgccattgccttgcttcattggagggtgtccgttcaata
  I S P S T L Y C H C L A S L E G V R S I
acgggagcccttaaaaaaaaatcttggcctggaattgacgatcttgccattaaaaaaaaa
  T G A L K K K S W P G I D D L A I K K K
ctgcctgatccggggggattaattcaaaaaaggggcaaaccaaaaaaaaaaaccggcttg
  L P D P G G L I Q K R G K P K K K T G L
gttgatttcctcatgggtgctggaataaagcccacctcaattgtcagttacaaccacttg
  V D F L M G A G I K P T S I V S Y N H L
gggaataatgatggcacgaacctttctgcgccgcaaacattccgatccaaggagatctcc
  G N N D G T N L S A P Q T F R S K E I S
aaaagcagcgtggtcgatgacatggtctcaagcaatgctatcctctacgagcctggcgag
  K S S V V D D M V S S N A I L Y E P G E
catcctgatcatgttgtcgtgattaagtatgtgccgtacgtcggagacagcaagagggcc
  H P D H V V V I K Y V O Y V G D S K R A
atggatgagtacacctcagagatcttcatggggggtaagaacaccatcgtgctgcacaac
  M D E Y T S E I F M G G K M T I V L H N
acctgcgaggactcgctccttgctgcaccaatcattcttgacctggtgctcctggccgag
  T C E D S L L A A P I I L D L V L L A E
ctcagcactaggattcagctgaaaggcgagggagaggagaaattccattccttccatcca
  L S T R I Q L K G E G E E K F H S F H P
gtggctaccatcctgagctacctcaccaaggcgccccttgttcctcctggcacaccagtg
  V A T I L S Y L T K A P L V P P G T P V
gtgaacgccctggcgaagcagagggctatgctcgagaacatcatgagggcctgcgttggg
  V N A L A K Q R A M L E N I M R A C V G
ctggcccctgagaacaacatgatcctggagtacaag
  L A P E N N M I L E Y K
```

The sequence has also been submitted to the GenBank (Accession Number AF 412340) and will be held confidential until Jun. 23, 2003.

| Comparison of aminoacid sequence of PINO1 with that of RINO1. | | | |
|---|---|---|---|
| RINO1: | 1 | MFIESFRVESPHRYGAAEIESDYQYDTTELVHESHDGASRWIVRPKSVRYNFRTTTIVP | 60 |
| | | MFIESFRVESPHRYGAAEIES+Y+YDTELVHESHDGASRW+VRPKSV+Y+FRT+TTVP | |
| PINO1: | 1 | MFIESFRVESPHRYGAAEIESEYRYDTTELVHESHDGASRWVRPKSVQYHFRTSTTVP | 60 |
| RINO1: | 61 | KLGVMLVGWGGNNGSTLTAGVIANREGISWATKDKVQQANYYGSLTQASTIRVGSYNGEE | 120 |
| | | KLGVMLVGWGGVVGSTLTAGVIA+REGISWATKDKVQQANYYGSLTQASTIRVGSYNGEE | |
| PINO1 | 61 | KLGVMLVGWGGNNGSTLTAGVIASREGISWATKDKVQQANYYGSLTQASTIRVGSYNGEE | 120 |
| RINO1: | 121 | IYAPFKSLLPMVNPDDLVFGGWDISNMNLADAMTRAKVLDIDLQKQLRPYMES------ | 173 |
| | | | IYAPFKSLLPMVNPDDLVFGGWDISNMNLADAMTRAKVLDIDLQKQLRPYMES |
| PINO1: | 121 | IYAPFKSLLPMVNPDDLVFGGWDISNMNLADAMTRAKVLDIDLQKQLRPYMESCLSLAS | 180 |
| RINO1: | 174 | MVPL--PGIYDPDVIAANQGSRANNVIKGTKKEQMEQIIKDIREFKEKSKVDKVVVLWTA | 231 |
| | | M+P P DP A SR ++ + + KDIREFKE +K+DK vvLWTA | |
| PINO1: | 181 | MIPTSSPLTRDP---ARTMSSRE------PRRSRWGRSSKDIREFKENNKMDKAVVLWTA | 231 |
| RINO1: | 232 | NTERYSN-VCVGLNDTMENLLASVDKNEAEISPSTLYAIACV-MEGIPFINGSPQNTFVP | 289 |
| | | NTERY+N +C+GL T ASVD+N+AEISPSTLY +EG+ I G+ + P | |
| PINO1: | 232 | NTERYNNCLCLGLM-TNGKPSASVDRNQAEISPSTLYCHCLASLEGVRSITGALKKKSWP | 290 |
| RINO1: | 290 | GLIDLAIKNNNCLI-GGDDFKSGQTKMKSVLVDFLVGAGIKPTSIVSYNHLGNNDGMNLSA | 348 |
| | | G+ DLAIK GG K G+ K K+ LVDFL+GAGIKPTSIVSYNHLGNNDG NLSA | |
| PINO1: | 291 | GIDDLAIKKKLPDPGGLIQIKRGKPKKKTGLVDFLMGAGIKPTSIVSYNHLGNNDGTNLSA | 350 |
| RINO1: | 349 | PQTFRSKEISKSNVVDDMVSSNAILYELGEHPDHVVVIKYVPYVGDSKRAMDEYTSEIFM | 408 |
| | | PQTFRSKEISKS+VVDDMVSSNAILYE GEHPDHVVVIKYVPYVGDSKRAMDEYTSEIFM | |
| PINO1: | 351 | PQTFRSKEISKSSVVDDMVSSNAILYEPGEHPDHVVVIKYVPYVGDSKRAMDEYTSEIFM | 410 |
| RINO1: | 409 | GGKSTIVLHNTCEDSLLAAPIILDLVLLAELSTRIQLKAEGEEKFHSFHPVATILSYLTK | 468 |
| | | GGK+TIVLHNTCEDSLLAAPIILDLVLLAELSTRIQLK EGEEKFHSFHPVPTILSYLTK | |
| PINO1: | 411 | GGKNTIVLHNTCEDSLLAAPIILDLVLLAELSTRIQLKGEGEEKFHSFHPVATILSYLTK | 470 |
| RINO1: | 469 | APLVPPGTPVVNALAKQRAMLENIMRACVGLAPENNMILEYK | 510 |
| | | APLVPPGTPVVNALAKQRAMLENIMRACVGLAPENNMILEYK | |
| PINO1: | 471 | APLVPPGTPVVNALAKQRAMLENIMRACVGLAPENNMILEYK | 512 |

On analysis it was revealed that the nucleotide sequences of the PINO1 gene is considerably non-identical resulting in gene-products in which the RINO1 mad PINO1 differ in the amino acid sequences for a stretch of about 110 in the mid-portion (between amino acids 173 to amino acids 320 of PINO1), the other parts of the genes bearing complete identity. The non-identical portion comprise of deletions/additions as well as conservative substitutions with two additional amino acids in case of PINO1 resulting in a protein having 512 amino acids in stead of reported 510 amino acids of RINO1.

Expression of RINO1 and PINO1 in Bacterial Expression Vectors:

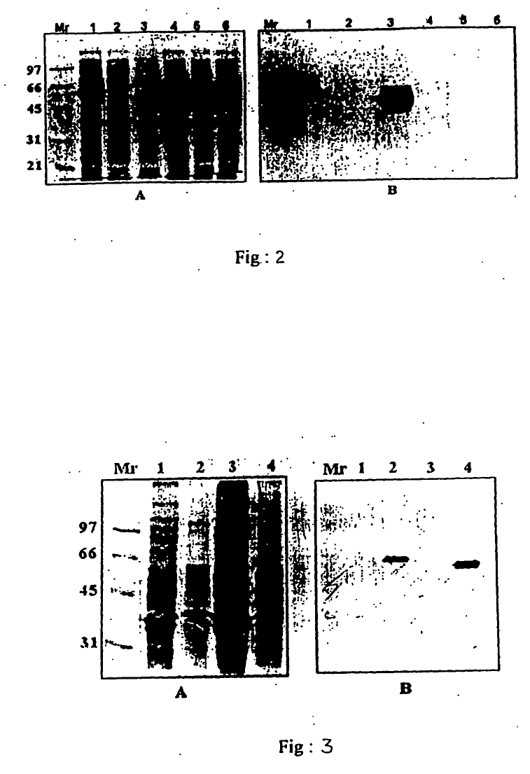

The cDNA for RINO1 and PINO1 were subcloned into suitable cloning sites of the bacterial expression vector pET 20B (+). The resulting plasmids were introduced into the host strain E. coli BL-21 (DE3). The bacteria were grown in LB medium up to A₆₀₀ of 0.5-absorbance unit and induced by 0.5 mM IPTG for 6 hours at 30° C. The bacteria were collected by centrifugation and lysed by sonication in a buffer containing 20 mM Tris-HCl, pH 7.5. 10 mM each of NH₄Cl and ME, 2 mM PMSF. The lysed extracts were centrifuged and protein from both soluble and membrane fractions were analyzed by 10% SDS-PAGE according to Lammeli (Nature, 227, 660-605, 1970) followed by western blot for immunodetection. The separated proteins were blotted onto PVDF membrane and the blot was probed with rabbit anti L-myo-inositol 1-phosphate synthase antibody (1:500) raised against purified recombinant L-myo-inositol 1-phosphate synthase of Entamoeba (Lohia et al, Mol. Biochem. Parasitol. 98, 67-79, 1999) or purified cytosolic L myo-inositol 1-phosphate synthage from Oryza leaves. Bound antibody was detected by the chemiluminiscence (kit from Amersham Life Sciences). Results of such experiments indicated that both RINO1 and PINO1 were expressed predominantly in the membrane fractions (FIG. 2, A & B, lanes 1 & 3).

Solubilization of Expressed RINO1 and PINO1 Proteins:

The expressed RINO1 and PINO1 proteins were solubilized from the pellet fractions in solubilization buffer (8M urea, 0.5 M NaCl, 20 mM Tris-HCl pH 7.5, 10 mM ME, 2 mM PMSF) kept for 30 minutes at room temperature. Solubilized samples were centrifuged at 15000 rpm for 30 minutes. Supernatant was taken and dialyzed serially in the same buffer with stepwise dilution of urea concentration from 8M to 2M. The solubilized samples were checked in SDS-PAGE and western blot for the

RINO1 and PINO1 proteins (FIG. 3, A & B, lanes 2 & 4). After solubilization, SDA-PAGE analysis revealed that the expressed protein was in soluble fraction (FIG. 3A) and was again confirmed by western blot analysis (FIG. 3 B).

Purification of the Solublized RINO1 and PINO1 Proteins:

The protein in the dialyzed sample was purified by DEAE Sephacel and Biogel A 0.5 by procedures earlier described from this laboratory (RayChaudhury et al., Plant Physiol., 115, 727-736,1997). Solubilized dialyzed sample was taken and loaded onto DEAE Sephacel column (20 ml bed volume). After two hours of absorption of the protein onto the column, the effluent was collected and then washed in buffer A containing 20 mM Tris-HCl, pH 7.5, 10 mM each of NH₄Cl and ME, 2 mM PMSF, 20% glycerol upto nearly 3 bed volume for elution of unbound protein and until the A₂₈₀ of the fractions approached 0. Bound proteins were eluted in 60 ml linear gradient of 0.01 to 0.25M NH₄Cl in buffer A. Fractions of 1 ml were collected at the rate of 0.4 ml/min. Fractions with inositol synthase activity were pooled, concentrated and dialyzed for 6 hr at 4° C. against 2 L change of buffer A. The dialyzed and concentrated, pooled DEAE fractions were loaded on a Biogel A 0.5 column, preequilibriated with 3 bed volumes of buffer A. Proteins were eluted with buffer A in fractions of 0.5 ml at a flow rate of 0.1 ml/min. Fractions containing inositol synthase activity were pooled, dialyzed against one 2 L change of 20 mM Tris-Cl (pH 7.5) 10 mM ME.

Biochemical Characterization of the Expressed RINO1 and PINO1 Proteins

The purified bacterially expressed RINO1 and PINO1 proteins were characterized for their biochemical properties (Table-1). Estimates of Km and Vmax values for the substrate (Glucose 6 phosphate) and co factor (NAD) were obtained with Biogel 0.5A purified recombinant synthase (s) using Line-Weaver Burk analysis. There is a difference between the Km values for glucose 6 phosphate of recombinant synthase of Oryza (RINO1) and Porteresia (PINO1). The lower Km values for glucose 6 phosphate for recombinant synthase of Porteresia (PINO1) suggest a higher substrate specificity compared to the Oryza recombinant synthase (RINO1). For both the cases optimum enzyme activity was at 37° C. whereas optimum pH for Porteresia recombinant synthase (PINO1) was 8.0 and the same for Oryza recombinant synthase (RINO1) was 7.5.

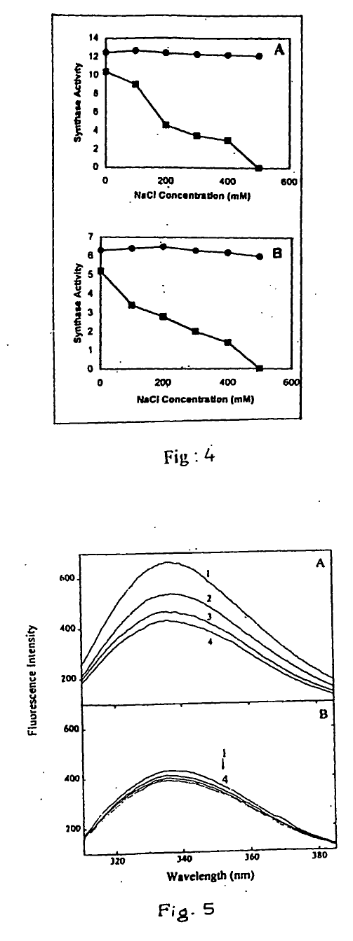

With respect to salt-sensitivity, RINO1 and PINO1 proteins differ a great deal. As in the case of the purified native enzymes (FIG. 4,A), the expressed recombinant RINO1 and PINO1 proteins exhibit similar characteristics with respect to salt-sensitivity/tolerance properties (FIG. 4, B). It is evident that both native and recombinant RINO1 proteins are sensitive to NaCl in vitro, whereas those of PINO1 are tolerant to salt under if vitra conditions upto a concentration of 500 mM NaCl adducing evidence that the expressed gene products of both retain their salt-sensitivity vis-à-vis salt-tolerance properties like the native enzyme proteins.

TABLE 1
Biochemical characterization of native and recombinant RINO1
and PINO1 proteins

| | PINO | | RINO | |
|---|---|---|---|---|
| CHARACTERS | Native | Recombinant | Native⁺ | Recombinant |

Km

| (1) G6P | 1.81 | mM | 2.5 | mM | 1.97 | mM | 3 | mM |
|---|---|---|---|---|---|---|---|---|
| (2) NAD | 0.153 | mM | 0.166 | mM | 0.14 | mM | 0.188 | mM |
| Vmax | | | | | | | | |
| (1) G6P | 0.08 | μmol^{-m} | 0.095 | μmol^{m} | 0.07 | μmol^{-m} | 0.072 | μmol^{-m} |
| (2) NAD | 0.12 | μmol^{-m} | 0.087 | μmol^{m} | 0.09 | μmol^{-m} | 0.068 | μmol^{-m} |
| pH optimum | 7.5 | | 8.0 | | 8.2 | | 7.5 | |
| Temperature | 35° C. | | 37° C. | | 35° C. | | 37° C. | |
| Optimum | | | | | | | | |
| Molecular weight | | | | | | | | |
| Native | ˜180 | kDa | ˜180 | kDa | ˜180 | kDa | ˜180 | kDa |
| Subunit | ˜60 | kDa | ˜60 | kDa | ˜60 | kDa | ˜60 | kDa |

⁺Data from RayChaudhury et al. (1997)

The invention is described in greater detail hereinafter, with reference to the accompanying drawings and examples, which are provided as mere illustrations of the invention and should not be construed to limit the scope thereof in any manner.

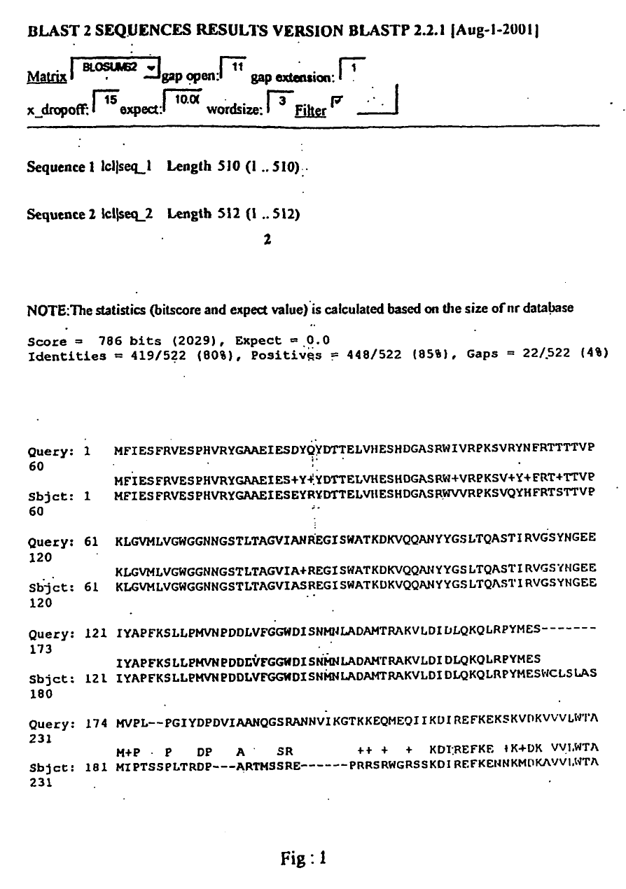

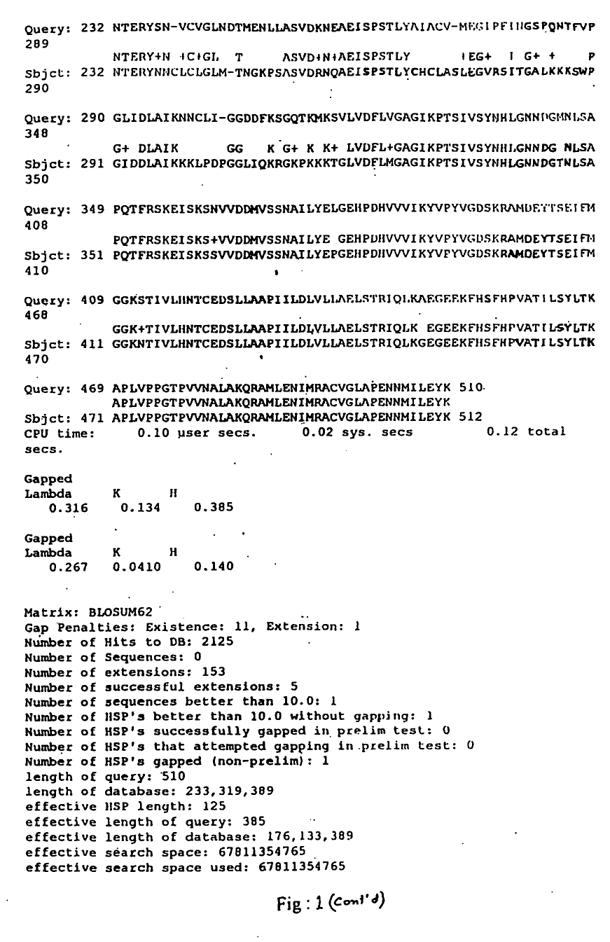

FIG. 1: (A) SDS-PAGE analysis of proteins of bacterially expressed RINO1 and PINO1; lanes 1&2-RINO1, induced and uninduced; lanes 3 & 4-PINO1 induced and uninduced; lanes 5 & 6-control-induced and uniduced. (1) Corresponding western blot of (A).

FIG. 2: (A) SDS-PAGE of proteins in pellet and supernatant fraction in the induced system after urea solubilization; lanes 1 and 2, pellet and supernatant of induced RINO1; lanes 3 & 4-pellet and supernatant of induced PINO1. (B) corresponding western blot of (A).

FIG. 3: Inositol synthase activity in presence of increasing NaCl concentration for purified native (A) and recombinant (B) enzymes.

FIG. 4: Tryptophan fluorescence of purified RINO1 (A) and PINO1 (B) proteins in increasing NaCl concentrations; tracing 1,2,3 & 4 correspond to 0, 100 mM, 20 mM and 400 mM NaCl in the system.

FIG. 5: Gel filtration pattern on Superose-12 of RINO1 and PINO1 proteins in absence and presence of 400 mM NaCl. (A) RINO1 without NaCl; (B) RINO1 with NaCl; (C) PINO1 without NaCl; (D) PINO1 with NaCl. Insets depict SDS-PAGE and immuno dot blots of indicated fractions.

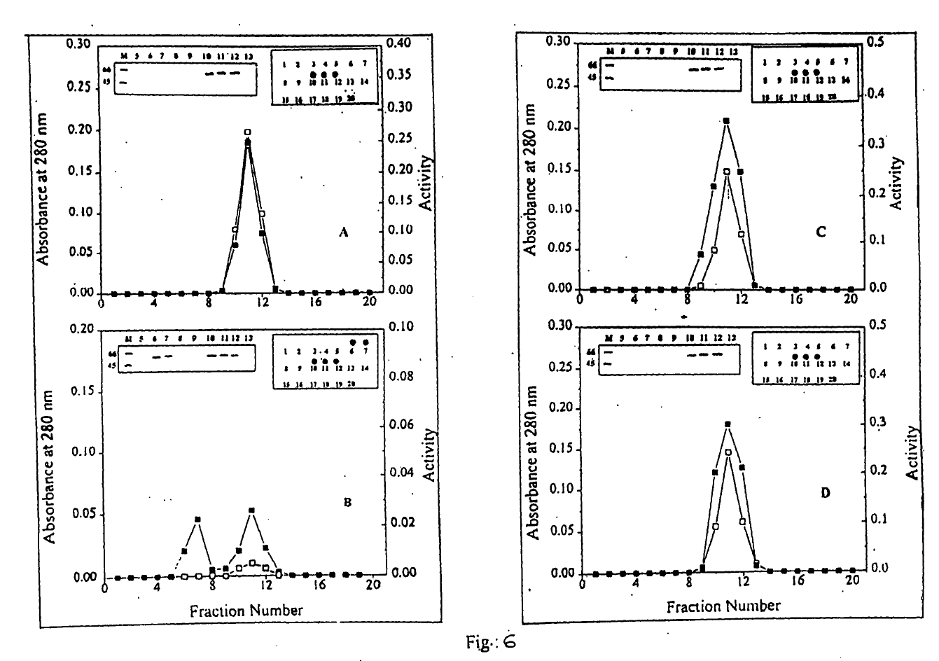

FIG. 6: Circular Dichroism spectra of RINO1 and PINO1 proteins.

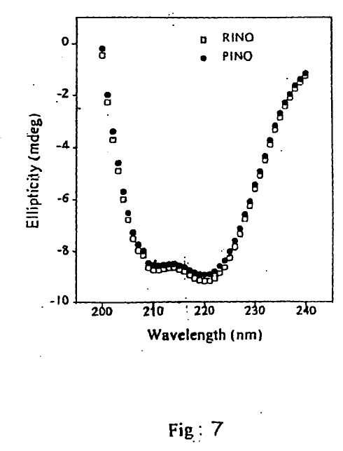

FIG. 7: Phenotype of nontransformed and PINO1-transformed tobacco plantlets grown with various concentration of NaCl in the growth media.

### EXAMPLE

Structural Studies of RINO1 and PINO1: Fluorescence, Circular Dichroism and Gel-Filtration Studies

In order to understand the structural basis of the differential behaviour of RINO1 and PINO1 towards salinity stress, we performed some fluorescence, Circular Dichroism (CD) and gel filtration experiments.

Tryptophan Fluorescence spectra of the recombinant preparations of the inositol synthase(s) from Oryza sativa (RINO1) and Porteresia coarctata (PINO1) are shown in Panel A and B respectively in FIG. 4. In absence of added salt, RINO1 shows significantly higher fluorescence intensity than PINO1 at the wavelength of maximum emission. The emission maxima in both the cases remain close to 336 nm. Fluorescence intensity of RINO1 is quenched significantly in presence of added salt whereas that of PINO1 is rather insensitive. It is also interesting to note that at salt concentration of over 600 mM, the fluorescence intensities of both RINO1 and PINO1 become comparable.

Progressive decrease of fluorescence intensity of RINO1 with increasing salt concentration indicates structural alterations. However, the emission maximum of RINO1 remains invariant as a function of increasing salt concentration meaning that the tryptophan environment remains unchanged. Tryptophan residues usually remain buried within the globular structure. Therefore the salt-induced changes do not interrupt the tryptophan microenvironment. It probably moves other protein segments closer to tryptophan to facilitate energy transfer and hence reduce intensity. The structure of PINO1 is stable to addition of salts. Since salts screen electrostatic interaction, there is considerable difference in the exposition of charged residues on the outer surface of RINO1 and PINO1.

The structure of RINO1 and PINO1 proteins in solution at the secondary level was probed by the Far-UV Circular Dichroism(CD) spectroscopy. The CD spectra of RINO1 and PINO1 proteins are almost identical in shape and show the characteristic bands (FIG. 5). The spectra were subjected to three-parameter secondary structure analysis Helix, sheet and random) by a non-linear curve fitting analysis according to K2D programme available from the K2D server on the Internet The analysis reveals that both RINO1 and PINO1 have very similar secondary structural elements having −25% α-helix, −25% β-sheet and 50% random plus other structures. Clearly the differences in the two proteins arise because of the different ways these secondary structural elements pack together.

In order to get ether insight into the nature of the structural changes in RINO1 due to addition of salt, we performed gel-permeation chromatography of RINO1 and PINO1 proteins both m presence and absence of added salt, the chromatograms are shown in FIG. 6. (A,B,C & D). It is seen that while RINO1 protein elutes as a single peak in absence of salt, addition of 400 mM NaCl leads to substantial reduction in the original peak and concurrent appearance of a high molecular weight fraction, suggesting oligomerization of the RINO1 protein by addition of NaCl. In contrast, PINO1 protein elutes at the same place corresponding the native trimeric association, both in absence and presence of NaCl. The activity data show that the trimeric form of the protein is enzymatically active although the oligomeric form (of the size of a tetramer) is inactive. Since oligomerization would affect mainly the protein surface and not the globular interior, the salt-sensitivity of the RINO1 protein may be explained by a suggested mechanism involving difference in ionic environments prevailing on the surface and a difference in hydrophobicity close to the surface when compared to the salt-tolerant PINO1 protein.

Phenotype of Tobacco Plants Transformed with PINO1 Gene During Salt-Growth

To determine whether introgression of PINO1 into plant system may help growing the plant in presence of salt, tobacco plants transformed with the PINO1 gene through the Agrobacterium-mediated procedure were raised. For this, the PINO1 gene was cloned into the plant expression vector, pCAMBIA 1301 and mobilized into tide Agrobacterium strain LBA 4404 by following standard procedures. Tobacco leaf discs, precultured in regeneration media were immersed in the suspension of Agrobacterium culture containing the PINO1-pCAMBIA construct for 1 hr and transferred back to the regeneration medium supplemented with cefotaxim and hygromycin. After shoot and root growth, the regenerated plantlets were transferred to culture vessels containing 0, 100, 200 and 400 mM NaCl for further growth. Control plantlets transformed with only pCAMBIA vectors were also grown in salts in similar way. A comparison of the plants (control and the PINO1-transformed) grown in presence of increasing amount of NaCl show that while the control plants exhibit loss of chlorophyll in presence of 200 mM NaCl, the PINO1 transformed plants exhibit no such loss of chlorophyll at the indicated salt concentration, although at 400 mM salt both types of plants fail to grow (FIG. 7). This might suggest that the PINO1 transformed plants are able to maintain the photosynthetic machinery in presence of NaCl at concentrations normally inhibitory to the growth of untransformed plants.

The above-mentioned experiments strongly 'suggest that the PINO1 gene sequence(s) may become a useful tool for production of transgenic crop plants tolerant to salt stress.

While the invention has been described in details and with reference to specific embodiments thereof, it will be apparent to one skilled in the art that various changes and modifications can be made therein without deviating or departing from the spirit and scope of the invention Thus the disclosure contained herein includes within its ambit the obvious equivalents and substitutions as well.

Having described the invention in detail with particular reference to the illustrative examples and comparative data given above, it will now be more specifically defined by means of claims appended hereafter.

We claim:

1. A salt-tolerant L-myo-inositol 1 phosphate synthase from Porteresia coarctata (PINO1) the nucleotide sequences and the deduced aminoacid sequence as given below (A)
```
A. Nucleotide and deduced aminoacid sequence of PINO1:
atgttcatcgagagcttccgcgtggagagcccgcacgtgcggtacggcgcggcggagatc
  M F I E S F R V E S P H V R Y G A A E I
gagtcggagtaccggtacgacactacggagctggtgcacgagagccacgacggcgcctcg
  E S W Y R Y D T T E L V H E S H D G A S
cgctgggtcgtccgccccaagtccgtccagtaccacttcaggaccagcaccaccgtcccc
  R H V V R P K S V Q Y H F R T S T T V P
aagctcggggtcatgctcgtggggtggggcggcaacaacggctcaacgctgacggctggg
  K L G V M L V G W G G N H G S T L T A G
gtcatcgccagcagggagggaatctcatgggcgaccaaggacaaggtgcagcaagccaac
  V I A S R E G I S W A T K D K V Q Q A N
tactatggctcactcacccaggcgtccaccatcagggtaggaagctacaacggggaggag
  Y Y G S L T Q A S T I R V G S Y N G E E
atctacgcgcctttcaagagcctcctgcccatggtgaaccctgatgaccttgtgttcggg
  I Y A P F K S L L P M V N P D D L V F G
ggctgggacattagcaacatgaacctggctgatgctatgaccagggccaaggtgctggac
  G W D I S N M N L A D A M T R A K V L D
attgatctgcagaagcagcttaggccttacatggagtcctggtgcctctccctggcatct
  I D L Q K Q L R P Y M E S W C L A L A S
atgatcccgacttcatcgccgctaaccagggatcccgcgcgaacaatgtcatcaagggaa
  M I P T S S P L T R D P A R T M S S R E
ccaagaaggagcagatggggcagatcatcaaaggacatcagggagttcaaggaaaataac
  P R R S R W G R S S K D I R E F K E N N
aaaatggacaaggcggtggtgttgtggactgcaaacactgaaaggtacaacaattgtctg
  K M D K A V V L N T A N T E R Y N N C L
tgtttgggcttaatgaccaatggaaaaccttctgcgtctgtggacaggaaccaggcggag
  C L G L M T N G K P S A S V D R S Q A E
atatcgccatcgacattgtattgccattgccttgcttcattggagggtgtccgttcaata
  I S P S T L Y C H C L A S L E G V R S I
acgggagcccttaaaaaaaaatcttggcctggaattgacgatcttgccattaaaaaaaaa
  T G A L K K K S W P G I D D L A I K K K
ctgcctgatccggggggattaattcaaaaaaggggcaaaccaaaaaaaaaaaccggcttg
  L P D P G G L I Q K R G K P K K K T G L
gttgatttcctcatgggtgctggaataaagcccacctcaattgtcagttacaaccacttg
  V D F L M G A G I K P T S I V S Y N H L
gggaataatgatggcacgaacctttctgcgccgcaaacattccgatccaaggagatctcc
  G N N D G T N L S A P Q T F R S K E I S
aaaagcagcgtggtcgatgacatggtctcaagcaatgctatcctctacgagcctggcgag
  K S S V V D D M V S S N A I L Y E P G E
catcctgatcatgttgtcgtgattaagtatgtgccgtacgtcggagacagcaagagggcc
  H P D H V V V I K Y V O Y V G D S K R A
atggatgagtacacctcagagatcttcatggggggtaagaacaccatcgtgctgcacaac
  M D E Y T S E I F M G G K M T I V L H N
acctgcgaggactcgctccttgctgcaccaatcattcttgacctggtgctcctggccgag
  T C E D S L L A A P I I L D L V L L A E
ctcagcactaggattcagctgaaaggcgagggagaggagaaattccattccttccatcca
  L S T R I Q L K G E G E E K F H S F H P
gtggctaccatcctgagctacctcaccaaggcgccccttgttcctcctggcacaccagtg
  V A T I L S Y L T K A P L V P P G T P V
gtgaacgccctggcgaagcagagggctatgctcgagaacatcatgagggcctgcgttggg
  V N A L A K Q R A M L E N I M R A C V G
ctggcccctgagaacaacatgatcctggagtacaag
  L A P E N N M I L E Y K
```

2. DNA sequence coding as claimed in claim 1 wherein the nucleotide sequences of PINO1 comprises of two additional amino acids resulting in a protein bearing 512 amino acids in comparison with RINO1, the L-myo-inositol 1-phosphate synthase from cultivated rice.

3. A process of obtaining a salt-tolerant L-myo-inositol 1-phosphate synthase gene comprising:
(i) Isolation of a full-length cDNA for the L-myo-inositol 1-phosphate synthase gene from the leaf of Porteresia coarctata by reverse transcription followed by polymerase chain reaction;
(ii) sequencing of the isolated L-myo-inositol 1-phosphate synthase gene.

4. A process as claimed in claim 3, wherein the isolated full-length cDNA of PINO1 is cloned into a suitable bacterial expression vector pET 20B(+) to produce expression plasmids.

5. A process as claimed in claim 4, wherein the said plasmids were introduced into the host strain E. coli BL-21 (DE 3) for obtaining an expressed PINO1 gene product.

6. A process as claimed in claim 5, wherein the expressed PINO1 proteins are solubilized in a solubilization buffer containing 8M Urea, 0.5 M NaCl, 20 mM Tris-HCl, pH 7.5,10 mM ME and 2 mM PMSF.
